SEQUENCE LISTING (2 sequences)

SEQ ID NO: 1 - protein, 183 aa, Hepatitis B virus
MDIDPYKEFGATVELLSFLPSDFFPSVRDLLDTAAALYRDALESPEHCSPHHTALRQAILCWGDLMTLATWVGTNLEDPASRDLVVSYVNTNVGLKFRQLLWFHISCLTFGRETVLEYLVSFGVWIRTPPAYRPPNAPILSTLPETTVVRRRGRSPRRRTPSPRRRRSQSPRRRRSQSRESQC

SEQ ID NO: 2 - protein, 149 aa, Hepatitis B virus
MDIDPYKEFGATVELLSFLPSDFFPSVRDLLDTAAALYRDALESPEHCSPHHTALRQAILCWGDLMTLATWVGTNLEDPASRDLVVSYVNTNVGLKFRQLLWFHISCLTFGRETVLEYLVSFGVWIRTPPAYRPPNAPILSTLPETTVV

(19) United States
(12) Patent Application Publication
(10) Pub. No.: US 2026/0284203 A1
(43) Pub. Date: Sep. 24, 2026

(54) REACTIVE CAPSID PROTEIN ASSOCIATED MOLECULES

(71) Applicants: The Trustees of Indiana University, Bloomington, IN (US); Door Pharmaceuticals, LLC, Indianapolis, IN (US)

(72) Inventors: Adam Zlotnick, Bloomington, IN (US); Charles Kim, Bloomington, IN (US); Michael Vannieuwenhze, Bloomington, IN (US); Jessica Gudorf, Bloomington, IN (US); William Turner, Bloomington, IN (US)

(21) Appl. No.: 19/469,091

(22) PCT Filed: Mar. 28, 2024
(86) PCT No.: PCT/US2024/022019

§ 371 Date: Sep. 25, 2025

Related U.S. Application Data
(60) Provisional application No. 63/492,760, filed on Mar. 28, 2023.

(51) Int. Cl. A61K 47/55; A61K 47/54; A61P 31/20

(52) U.S. Cl. CPC A61K 47/55; A61K 47/545; A61P 31/20

(57) ABSTRACT

Provided are chemical compounds composed of a hepatitis B virus (HBV) targeting ligand linked to a nucleic acid reactive molecule. The HBV targeting ligand can include capsid protein associated molecule. The molecules direct the nucleic acid reactive molecules to the HBV particles to treat and facilitate a cure for chronic HBV infection. The present disclosure also provides methods of making such compounds and pharmaceutical compositions containing the compounds.

### CROSS-REFERENCE TO RELATED APPLICATION

The present application claims priority to U.S. Provisional Application No. 63/492,760, filed on Mar. 28, 2023, and entitled “REACTIVE CAPSID PROTEIN ASSOCIATED MOLECULES,” the entire disclosure of which is expressly incorporated by reference herein.

### STATEMENT OF GOVERNMENTAL RIGHTS

This invention was made with government support under AI144022 awarded by National Institutes of Health. The Government has certain rights in the invention.

### FIELD OF THE INVENTION

The present disclosure relates generally to chemical constructs, such as antiviral constructs composed of a capsid assembly modulator (CAM), a linker, and a reactive moiety. Also disclosed are methods of using such antiviral constructs to treat viral infections, such as hepatitis B virus (HBV), among other conditions.

### BACKGROUND

Chronic Hepatitis B Virus (HBV) infection is a world health problem. More than 240 million people are afflicted with chronic infection which can lead to liver failure, cirrhosis, and hepatocellular carcinoma. WHO Globalhepatitis report; World Health Organization, 2017. Today, there are no widely curative treatments for chronic HBV infection. Many patients rely on a lifetime therapy of nucleotide analogs (NUCs), and most will experience viral rebound if they stop taking NUCs. There is a great need for developing better therapeutics.

Core protein (Cp) directed antivirals, such as capsid assembly modulators (CAMs) have emerged as a potential solution to the HBV therapy problem. However, one of the factors that still makes developing a functional cure so difficult is due to the persistence of HBV's covalently closed circular DNA (cccDNA). During chronic infection, HBV will have established a pool of cccDNA in the host hepatocyte's nucleus, which will allow for a steady stream of viral transcripts and virion assembly. Seeger, C. and Mason, W. S., Virology 672: 479-480, 2015. Clearing cccDNA has been a challenging task since newly synthesized virions can infect a cell. Also, DNA-containing virions can be secreted and go on to infect more host cells. Prange, R., Biol Cell 2022, 114, 325. Some CAMs can dissociate DNA-filled capsids, and some have been shown to reduce cccDNA levels when cells have been pretreated with drugs. Guo, F., et al., PLoS Pathog 2017, 13, e1006658; Berke, J. M., et al., Antimicrob Agents Chemother 2017, 61; Berke, J. M., et al., Antimicrob Agents Chemother 2020, 64, pii:e02439; and Mani, N., et al., Animicrob Agents Chemother 2018.

However, CAMs, like NUCs, are not curative. They suppress virus expression and inhibit new infection, but they do not eliminate infection because they do not attack HBV nucleic acid, particularly the nuclear copy of the viral genome, which acts as the stable template for HBV expression. There remains a need for improved treatment of chronic HBV infection. In particular, there exists a need for antiviral treatments and HBV treatments that can clear established cccDNA. The present disclosure addresses these long-standing and unmet needs.

### INCORPORATION BY REFERENCE

Each patent, publication, and non-patent literature cited in the application is hereby incorporated by reference in its entirety as if each was incorporated by reference individually, and as if each is fully set forth herein. However, where such reference is made, and whether to patents, publications, non-patent literature, or other sources of information, it is for the general purpose of providing context for discussing features of the invention. Accordingly, unless specifically stated otherwise, the reference is not to be construed as an admission that the document or underlying information, in any jurisdiction, is prior art, or forms part of the common general knowledge in the art.

### SUMMARY OF THE INVENTION

A first aspect of the invention includes a novel antiviral compound for directed delivery of HBV antiviral agent to HBV nucleic acid bound to capsid protein.

A second aspect of the invention includes compositions, such as pharmaceutical compositions including a compound of any of the following embodiments.

A third aspect of the invention includes methods of making compounds for directed delivery of an HBV antiviral agent to HBV nucleic acid bound to capsid protein.

A first embodiment is a compound for directed delivery of HBV antiviral agent to HBV nucleic acid bound to capsid protein, including a) an HBV capsid assembly modulator (CAM); b) a linker; and c) a nucleic acid disrupting reactive moiety, where the linker connects the CAM and the reactive moiety.

A second embodiment is a compound where the CAM interacts with, such as hydrogen bonds to, residues W102, S121, and T128 of the capsid protein.

A third embodiment is a compound where the linker has a length of about 0 to 20 Å, inclusive.

A fourth embodiment is a compound that has a linker of the following formula (I): FG₁-R-FG₂ (I), where FG₁ and FG₂ may independently include COOH, OH, and NH₂ and R includes an alkyl group of 2 to 5 methylenes, inclusive.

A fifth embodiment is a compound that has a linker of the following formula (I): FG1-R-FG2 (I), where FG1 and FG2 may independently include COOH, OH, and NH2 and R includes 1 to 6 polyethylene glycol units, inclusive.

A sixth embodiment is a compound that further includes an amide spacer positioned between the CAM and the linker.

A seventh embodiment is a compound where the CAM is connected directly to the nucleic acid disrupting reactive moiety.

An eighth embodiment is a compound where the nucleic acid disrupting reactive moiety is selected from an alkylating agent or a reactive oxygen species generator.

A ninth embodiment is a compound where the alkylating agent is a chemotherapeutic molecule selected from the group consisting of chlorambucil, altretamine, mitomycin C, cisplatin, phleomycin, and analogs thereof.

A tenth embodiment is a compound where the reactive oxygen species generator includes a polyphenol, a dihydroxy phenol, a trihydroxy phenol, proteocatachuic acid, gallic acid, 2,3,4-trihydroxybenzoic acid, a dihydroxybenzoic acid, or a combination thereof.

An eleventh embodiment is a compound where the dihydroxybenzoic acid includes 2,3-dihydroxybenzoic acid, 2,4-dihydroxybenzoic acid, 2,5-dihydroxybenzoic acid, 2,6-dihydroxybenzoic acid, 3,4-dihydroxybenzoic acid, 3,5-dihydroxybenzoic acid, or a combination thereof.

A twelfth embodiment is a compound where the CAM is selected from the group consisting of tetrahydropyrazolopyrazine, sulfamoyl benzamide (SBA), pyrazolopyridine, phenylpropenamide (PPA), heteroaryldihydropyrimidine (HAP), pyrole benzamide, sulfonylpiperdine carboxamide, dibenzothiazepine (DBT), pyrrole carboxamides, and analogs thereof.

A thirteenth embodiment is a compound that has or includes the structure

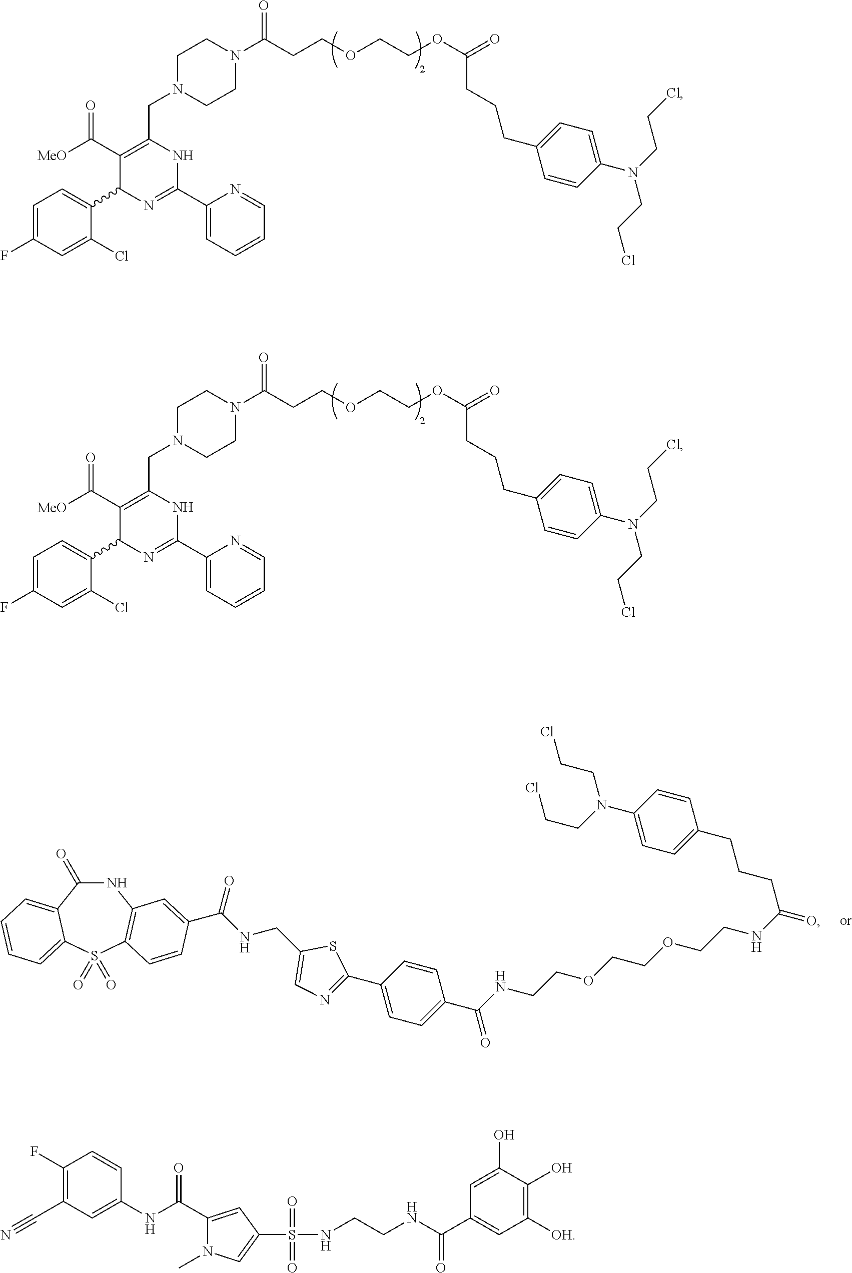

A fourteenth embodiment is a pharmaceutical composition including the compound of any of the preceding embodiments and a pharmaceutically acceptable excipient.

A fifteenth embodiment is a method of making a compound for directed delivery of an HBV antiviral agent to HBV nucleic acid bound to capsid protein, including coupling an HBV capsid assembly modulator (CAM) to a first end of a linker and attaching the nucleic acid disrupting reactive moiety to a second end of the linker, where the linker separates the CAM from the nucleic acid disrupting reactive moiety.

A sixteenth embodiment is a method where the coupling step includes an amide coupling reaction.

A seventeenth embodiment is a method where the attaching step includes an esterification reaction.

An eighteenth embodiment is a method where the linker is absent, and the CAM is directly attached to the nucleic acid disrupting reactive moiety.

A nineteenth embodiment is a method for delivering nucleic acid disrupting reactive molecule to an HBV capsid, the method comprising providing an antiviral compound having a (CAM), a linker, and a nucleic acid disrupting reactive moiety to cells infected with HBV, where the compound directs the reactive moiety to the HBV capsid protein through the CAM portion of the compound, interacts with the pocket of the HBV capsid protein, and places the nucleic acid disrupting reactive moiety in proximity to the HBV nucleic acid within the HBV capsid.

A twentieth embodiment is a method for delivering a nucleic acid disrupting reactive molecule to an HBV infected cell, the method comprising providing an antiviral compound having a (CAM), a linker, and a nucleic acid disrupting reactive moiety to cells infected with HBV, where the CAM directs the nucleic acid disrupting reactive moiety to an HBV core in the infected cell.

A twenty-first embodiment is a method for delivering a nucleic acid disrupting reactive molecule to an HBV infected cell, the method comprising providing an antiviral compound having a (CAM), a linker, and a nucleic acid disrupting reactive moiety to cells infected with HBV, where the CAM directs the nucleic acid disrupting reactive moiety to an HBV episomal cccDNA in the infected cell.

### BRIEF DESCRIPTION OF THE FIGURES

The accompanying drawings are included to provide a further understanding of the disclosure and are incorporated in and constitute a part of this specification, illustrate embodiments, and together with the description, serve to explain the principles of the disclosure.

FIG. 1 is a schematic showing structural alignment of the CAMs listed in Table 1 within the helix 5 pocket.

FIG. 2 shows modelling of the superpositions for each of the CAMs listed in Table 1 as aligned in FIG. 1 separately. The protein subunits are covered with a molecular surface, where the pocket subunit is shown on the left side of the image, and the capping subunit is shown on the right side of the image to provide ease in visualizing the CAM structure by covering the protein subunits with a molecular surface. A CAM-linker-fluorophore construct, HAP-TAMIRA, is used as a comparator for visualization of the position in the pocket.

FIG. 3 shows the structures of exemplary CAM scaffolds with the linker attachment site circled.

FIG. 4 shows a schematic illustrating modular construction of rCAM target molecules, utilizing a DBT CAM structure, amide spacer, linker, and exemplary warheads chlorambucil or gallic acid. Pg on the linker denotes a protecting group.

FIG. 5A shows the structure of exemplary warhead chlorambucil, an anticancer mustard, with the point of attachment for the linker circled.

FIG. 5B shows the structure of exemplary warhead gallic acid, a reactive oxygen species generator, with the point of attachment for the linker circled.

FIG. 6A and FIG. 6B are images from denaturing agarose gel electrophoresis depicting activity levels of alkylating agents, chlorambucil (Chlor), mitomycin C (Mito), cisplatin (Cisp), phleomycin (Phleo), and altretamine (Altre) on pgRNA copy number.

FIG. 6C and FIG. 6D are bar graphs depicting evidence of alkylation by chlorambucil, altretamine, mitomycin C, cisplatin, and phleomycin on pgRNA copy number using RT-qPCR.

FIG. 7 shows a reaction scheme for synthesizing an exemplary rCAM compound, HAP-PEG2-CBL, wherein HAP is the CAM, PEG2 is the linker and CBL is the reactive moiety.

FIG. 8 shows a reaction scheme for synthesizing DPC-323, an exemplary rCAM having a Dibenzothiazepine CAM and a Chlorambucil reactive moiety.

FIG. 9 is a gel electrophoresis image of the alkylating activity of HAP-PEG2-CBL (HP2C) and HAP-PEG4-CBL (HP4C) rCAM constructs.

FIG. 10A shows Transmission Electron Microscopy (TEM) images of Cp149 dimers in the absence of a rCAM; FIG. 10B shows TEM images of Cp149 dimers assembled with rCAM HAP-PEG2-CBL; and FIG. 10C shows TEM images of Cp149 dimers assembled with rCAM HAP-PEG4-CBL.

FIG. 11 shows physical characterization of rCAM HAP-PEG2-CBL. Rₜ=11.241 minutes (analytic RP HPLC, 10%→90% MeCN in H₂O with 10% of 1% TFA (aq); gradient over 10 minutes, 1 mL/min, 254 nm); ¹H NMR (500 MHz, Chloroform-d) δ 8.54 (dd, J=5.0, 1.5 Hz, 1H), 8.18 (d, J=7.9 Hz, 1H), 7.74 (td, J=7.8, 1.7 Hz, 1H), 7.36 (ddd, J=7.5, 4.9, 1.2 Hz, 1H), 7.27 (dd, J=8.6, 6.1 Hz, 1H), 7.13 (dd, J=8.6, 2.6 Hz, 1H), 7.09-7.03 (m, 2H), 6.89 (td, J=8.3, 2.6 Hz, 1H), 6.66-6.58 (m, 2H), 6.26 (s, 1H), 4.27-4.19 (m, 3H), 4.10 (d, J=17.3 Hz, 1H), 3.81 (t, J=6.8 Hz, 2H), 3.69 (d, J=6.6 Hz, 6H), 3.66-3.55 (m, 14H), 2.66 (t, J=6.8 Hz, 2H), 2.65-2.50 (m, 6H), 2.34 (t, J=7.5 Hz, 2H), 1.96-1.84 (m, 2H), 1.38 (s, 2H); TOF-MS (ESI+) m/z [M+H]⁺ calculated for C₄₃H₅₃Cl₃FN₆O₇ (M+H)⁺ 891.3001; found 891.3000.

FIG. 12 shows a reaction scheme for synthesizing DP41-0012, an exemplary rCAM having a pyrrole CAM and a gallic acid reactive moiety.

FIG. 13 shows physical characterization of exemplary rCAM DP41-0012 using LC/MS.

FIG. 14 is a bar graph showing relative peak HBV capsid volume in an assay evaluating the effect of the CAMs bersacapavir and NVR-3-778, exemplary rCAM DP41-0012, and controls on HBV capsid assembly.

FIG. 15 shows a gel electrophoresis image visualizing nicked, linearized, and supercoiled

### DETAILED DESCRIPTION

In some aspects, described herein is a new class of antiviral that will specifically attack nucleic acid that is bound to capsid protein. We refer to these compounds as reactive Capsid protein Associated Molecules, or rCAMs. Each rCAM is defined by three components: (i) a capsid protein binding component (CAM) to target the HBV capsid protein (Cp), (ii) a linker to connect the Cp binding component to a reactive moiety, and (iii) a nucleic acid-disrupting reactive moiety (also referred to as a “reactive moiety” or “warhead”) that can cleave, alkylate, or crosslink local nucleic acid.

The role of the CAM is to localize the reactive moiety to HBV capsids and virus capsid protein (HBc)-DNA complexes. The reactive moiety should alkylate, incorporate nonsense mutations, breaks, or otherwise prevent replication of viral nucleic acid. The linker must be long enough so that the reactive moiety does not interfere with binding to the capsid protein. The linker may be a separate component of the rCAM or the linker may be a portion of the CAM molecule itself that serves the function of a linker. Details of the linker depend in part on the affinity of the CAM for Cp and the mechanism of the reactive moiety.

The targeting CAM itself is intended to have negligible antiviral activity while the warhead component creates damage in the viral nucleic acid, which can lead to error prone repair of viral DNA or simply render the viral nucleic acid unusable. Because CAMs specifically bind to capsid protein, these rCAM molecules can specifically target nucleic acid-filled particles and their content, minimizing non-specific interactions with host genome. This CAM-facilitated localization of the reactive moiety to viral nucleic acid, allows the use of very low doses of the reactive moiety, such as a chemotherapeutic, and favors chemical attack on the virus and viral DNA in the nucleus of the host cell.

By chemically modifying DNA in a virus particle, the disclosed rCAMs will prevent new infection. Additionally, by chemically modifying viral DNA in the nucleus of the host cell the disclosed rCAMs will prevent replication of new virus. The disclosed molecules are intended to treat and to facilitate a cure for chronic HBV infection.

### Hepatitis B Virus Infection Cycle

In an HBV infection, the virus enters a cell and delivers its genome to the host nucleus, where it is “repaired” and chomatinized by the host and then serves as the template for viral RNAs. A full length, terminally redundant RNA and a copy of newly translated viral reverse transcriptase is packaged in an immature viral capsid protein. In this immature core, the RNA is reverse transcribed to yield the circular dsDNA genome found in the mature core. Mature cores may become enveloped and get secreted from the cell to infect the next cell or organism, or it may be recycled back to the nucleus.

At each step in the viral replication cycle, the virus capsid protein (HBc or Cp) is specifically associated with viral nucleic acid. HBc is a small homodimeric protein that assembles into a 120-dimer icosahedral capsid. In the incoming virus, the HBc capsid encases the genome and, by interaction with host proteins, directs it to the nuclear pore; presumably by interactions at the pore, it releases the genome into the nucleus. Gallucci and Kann, Viruses 9:21, 2017; Osseman and Kann, Methods Mol Biol 1540:37-51, 2017. In the nucleus, HBc helps to recruit host machinery involved in chromatinization and remains closely associated with the viral genome, even after repair. Lucifora, et al., JHEP Rep 3:100330, 2021; Locatelli, et al., Cell Mol. Gastroenterol Hepatol 14:527-551, 2022. HBc specifically assembles around viral RNA when forming an immature core and acts as a metabolic container for reverse transcriptase. Nassal, M., Virus Res 134:235-49, 2008; Tavis, et al., J Virol 68:3536-43, 1994; Pollack, J. R. and Ganem, D., J Virol 68:5579-87, 1994; Bartenschlager, R. and Schaller, H., EMBO J 11:3413-3420, 1992; Wang, J. C., et al., Proc Natl Acad Sci USA 111:11329-34, 2014; Nair, S. and Zlotnick, A, J Virol 92: e02190-17, 2018.

As used herein, the terms “treating” or “treatment” refer to a desired biological or pharmacological effect as described herein, as well as any one or more of: (a) preventing a disorder from occurring in a subject who may be predisposed to the disorder but has not yet been diagnosed with it; (b) inhibiting a disorder, e.g., interfering with the replication of HBV, and (c) relieving a disorder, e.g., causing regression of HBV-mediated liver fibrosis. Other such measurements, benefits, and surrogate or clinical endpoints, alone or in combination, will be understood to one of ordinary skill based on the teachings herein and the knowledge in the art.

As used herein, the terms “therapeutically effective amount” or “therapeutically effective dose,” e.g., of a composition comprising a disclosed rCAM compound, refers to an amount that is effective to achieve a desired therapeutic result. Exemplary measures of therapeutic efficacy in the context of treating viral infections include determining viral load, evaluating serological responses to viral infection, and evaluating indirect measures of viral infection, e.g., liver fibrosis in the context of HBV infection. Therapeutically effective amounts will typically depend upon the IC₅₀ and safety profile of the specific agent being administered.

### Compounds

In some aspects, provided herein are reactive CAM compounds comprising a capsid assembly modulator (CAM), a linker, and a reactive moiety. Herein, such compounds may be referred to simply as an “rCAM” or as “rCAMs.” In some embodiments, the linker connects the CAM to the reactive moiety. The CAM can localize the disclosed compounds to a viral capsid and/or a complex of viral DNA and capsid protein. In preferred examples, the linker has sufficient length to allow the reactive moiety attached to the CAM to reach viral nucleic acid. The reactive moiety can prevent the replication of viral nucleic acid, by any mechanism, including, e.g., DNA damaging alkylation, gene mutations, DNA double-stranded break formation, or incorporation of chain-terminating nucleoside/nucleotide analogues.

### Capsid Assembly Modulators (CAMs)

In the effort to develop antivirals, HBc assembly and disassembly have been identified as target reactions. See, e.g., Viswanathan, U., et al., Antiviral Res 182:104917, 2020; Zlotnick, A., et al., Anitiviral Res 121:82-93, 2015; Venkatakrishnan, B. and Zlotnick, A., Annual Reviews of Virology 3:429-451, 2016. Capsid Assembly Modulators (CAMs) are assembly agonists that speed up HBc assembly and stabilize HBc-HBc interactions. By doing so, in an infected cell, CAMs favor spontaneous HBc assembly, leading to empty and aberrant particles, rather than genome-nucleated assembly.

To date, published CAM structures bind to the same site at the interface between two dimers (the interdimer contact). See, e.g., Bourne, C., et al., J Virol 80:11055-11061, 2006; Katen, S. P., et al., Structure 21:1406-16, 2013; Venkatakrishnan, B., et al., J Virol 90:3994-4004, 2016; Schlicksup, C. J., et al., ACS Chem Biol 15:1708-1717, 2020; Schlicksup C. J. and Zlotnick, A., Curr Opin Virol 45:43-50, 2020; Klumpp, K, et al. Proc Natl Acad Sci USA 112:15196-201, 2015; Zhou, Z., et al., Sci Rep 7:42374, 2017; Kang, J. A., et al., Nat Commun 10:2184, 2019; Kuduk, S. D., et al., Bioorg Med Chem Lett 39:127848, 2021; Cole, A. G., et al., RSC Med Chem 13:343-349, 2022, each incorporated herein by reference.

The CAM binding site, at the junction of two dimers, is comprised of a shallow pocket in one dimer capped by a second dimer. All known CAMs bind to this site. Filling this pocket with a hydrophobic molecule can thermodynamically stabilize the protein-protein interaction, favoring capsid assembly. Filling the pocket with mutations (e.g., V124W) also stabilizes the interaction and favors assembly compared to wildtype protein (Tan et al., J Virol. 2015 Mar. 15; 89(6): 3275-3284). The same thermodynamic argument holds that any molecule that speeds up assembly must bind to product, that is capsid.

The major features of CAMs and the CAM site is molecular complementarity. The structures of Cp149 capsids incubated with CAMs have been investigated using X-ray crystallography (pdb code 2G34, 4G93 and 5D7Y). The investigated CAMs bind to a specific pocket, which is at the inter-dimer interface and consists of a concave interface made up of residues from one chain—F23, P25 of loop 2, D29, L30, T33, L37 from helix2, W102, I105, S106, T109, F110 of helix4, Y118 and F122 of helix5, 1139, L140, S141 of loop6 and cap from the other chain—V124, W125, R127 from helix5 and T128, P129, Y132, R133, P134 of loop6 (Viswanathan et al., Antiviral Research, 17 Aug. 2020, 182:104917).

Most CAMs, but not all, hydrogen bond to W102, S121, and T128. Residues forming the site include all or most of the following residues: F23, P25, D29, L30, T33, W102, I105, S106, F110, Y118, 1139, and L140 from the pocket subunit and from the capping subunit residues S121, V124, W125, R127, T128, Y132, and P134. Residues listed here are for genotype D, but residues are highly conserved across Genotypes A-E. The HBV capsid protein residues involved in CAM binding are described, e.g., by Zhou et al., Sci Rep. 2017; 7: 42374, Nicklasch et al., Biomedicines. 2021 November; 9(11): 1577, and Viswanathan et al., Antiviral Research, 17 August 2020, 182:104917. Capsid protein sequences are available to one of skill in the art, e.g., by retrieval from a sequence database such as UniProt. See, e.g., UniProt P03147 (HBV-D), which describes the capsid sequence of Hepatitis B virus genotype D subtype adw (isolate United Kingdom/adyw/1979). SEQ ID NO:1 and SEQ ID NO:2 show HBV core protein sequences Cp183, and Cp149 for adyw strains.

In the context of a capsid, the CAM binding site opens to the capsid lumen. This opening is framed by a group of amino acids generally including residues 109, 110, 118, 139, 140, and 141 of the pocket subunit, and residues 120, 121, 124, 125, and 134. Beyond this opening, CAM has negligible interaction with the capsid protein. More importantly, in a nucleoprotein complex, beyond this opening lies viral RNA or DNA.

In the absence of a CAM, the dimer-dimer interaction includes a large empty pocket and has weak association energy (Ceres, P. and Zlotnick, A., Biochemistry 41:11525-31, 2002). Filling the pocket with a CAM increases buried non-polar surface area and strengthens association energy (Bourne, C., et al., J Virol 82:10262-10270, 2008). This pocket has a “pocket” surface comprised of helices 2, 3, and 5 and a “capping” surface on the adjacent dimer comprised almost entirely of helix 5. In capsid structures, it can be seen that the pocket opens to the interior of the capsid. This opening allows CAMs to soak into preformed capsids. Of note, when the CAM favors a non-icosahedral geometry (i.e., by over-filling the CAM pocket) it can destabilize capsids causing dissociation. See, e.g., Ko, C., et al., Antimicrob Agents Chemother 64, e01440-19 2019, Berke, J. M., et al., Antimicrob Agents Chemother 61:e00560-17, 2017; and Berke, J. M., et al., Animicrob Agents Chemother 64:pii:e02439-19, 2020. The HBV capsid protein has no similarity to any human protein, providing a basis for the specificity of CAMs.

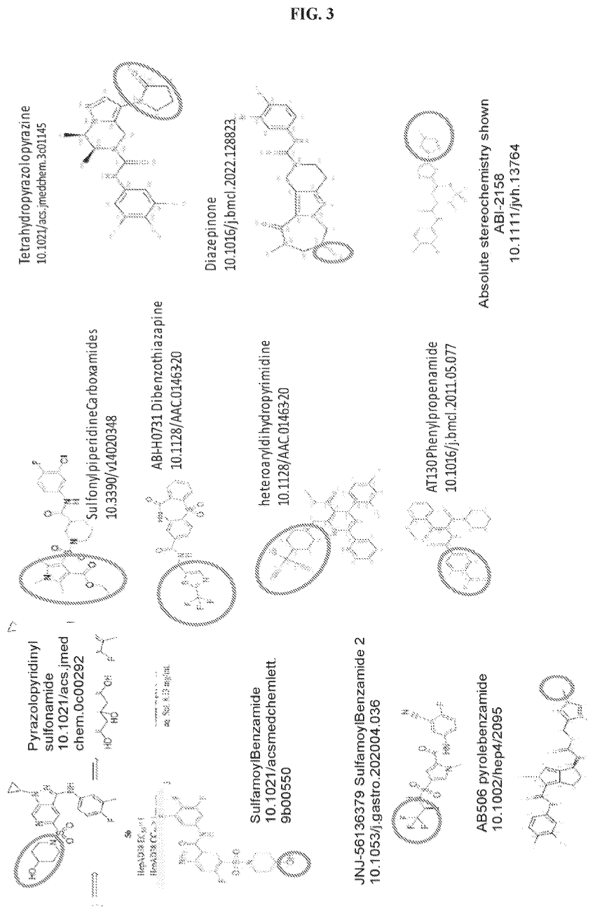

As shown in FIG. 3, CAM molecules can be based on a wide variety of scaffolds. A classical CAM molecule acts by initiating and accelerating capsid assembly in the absence of viral pgRNA and Polymerase, resulting in empty and/or aberrant capsids. This consumes capsid protein without providing infectious particles. A subset of CAMs will bind to infectious DNA-filled capsids and prevent them from infecting cells by inducing capsid fracture at an inappropriate times. Germane to this discovery, both mechanisms indicate that CAMs bind to capsids. This is also demonstrated by crystal and cryo-EM structures where CAMs have been added to pre-formed capsids.

An rCAM can be based upon any CAM known in the art. However, there are two critical characteristics of a CAM that make it suitable for development as an rCAM: the CAM must have high affinity for the CAM pocket and the CAM structure must be such that it can extend though the mouth of the CAM pocket. An additional characteristic, the on- and off-rates, can be important depending on the reactive moiety.

For rCAMs to function, they place the reactive moiety in the proximity of viral nucleic acid. That is, the rCAM must bind to the CAM site. Ideally the dissociation constant for binding (KD) should be less than 0.2 μM. Measuring binding activity may be performed by typical approaches such as isothermal titration calorimetry (ITC) or microscale thermophoresis. Binding can be approximated using an rCAM in which the reactive moiety is replaced by a fluorophore (e.g. Alexa fluor 488) and binding determined by change in fluorescence intensity or by sedimentation assay. In the sedimentation assay, the fluorophore labeled rCAM (typically at 1 μM) is mixed with capsid (also 1 μM with respect to dimer concentration), the absorbance at 488 nm of the sample is measured, after 1 h incubation the sample is centrifuged at 150,000×g for 30 m, and then the amount of labeled rCAM remaining in solution is determined by absorbance. The KD is determined by the following equation:

KD=[bound rCAM]/([free rCAM][free Cp]).

In this equation, brackets indicate concentration. The concentration of free rCAM is the amount of rCAM remaining in solution after centrifugation. The [bound rCAM] is the total rCAM less the amount bound. The free Cp is the initial Cp concentration less [bound rCAM].

The position of the reactive moiety is critical with respect to the CAM site and the CAM moiety geometry. CAM skeletons broadly overlap in terms of how they fit into the CAM site. The way that many fill the site includes a process extending towards the site opening. Structural examples include HAPs including GLS4, DBTs including Vebicorvir, SBAs including NVR3-778 and Bersacapavir, tetrahydrocyclopentapyrroles (PDB: 7S76), and Oxadiazepinones (PDB: 7K5M) (Kuduk et al., Bioorganic & Medicinal Chemistry Letters (2021) 39, 127848). These extensions may end with a functional group that can be connected to a linker. One skilled in the art can incorporate such a functional group. For example, HAP13 is an analog of highly active HAP12 (Bourne et al., J Virol. 2008 October; 82(20): 10262-10270), where the HAP12 morpholine is replaced by a piperazine. The resulting CAM is less active, but the reactive secondary amine of the piperazine has been used to connect to fluorophores (U.S. Pat. No. 10,006,913) and, in this embodiment, to reactive warheads of alkylating agents, such as chlorambucil, and reactive oxygen species generators, such as gallic acid.

If an rCAM has a reactive moiety that is an alkylating agent, such as a mustard like chlorambucil, then it is necessary for the warhead to contact the nucleic acid. In such a case either the linker must be long enough for the rCAM to extend from the pocket to the bound nucleic acid (ca. 20 Å) or the rCAM must stochastically bind and dissociate with the capsid protein on a biologically relevant time scale. That is the half-life for the dissociation reaction should be less than 30 minutes. A short half-life is exemplified by the SBA NVR3-778.

If an rCAM has a reactive moiety that generates freely diffusing reactive oxygen species (ROS) such as peroxide, peroxide radical, or hydroxy radical, the rCAM may bind the CAM site with very high affinity and/or with a very slow off-rate. That is, that once the rCAM is bound, it stays bound. The reactive moiety in such rCAMs may be a trihydroxy phenol, such as gallic acid (3,4,5 trihydroxy benzoic acid), or a dihydroxy phenol, such as protocatechuic acid (3,4 dihydroxy benzoic acid).

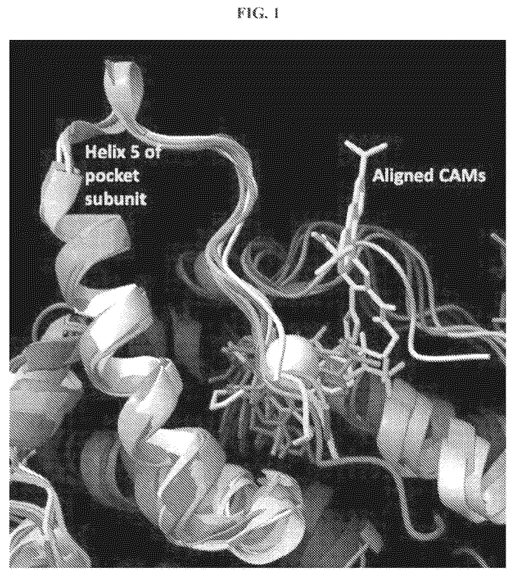

All published CAM structures show binding to a common pocket that opens to the capsid interior. Table 1 provides an exemplary number of CAM-HBc complex structures from the protein data bank (PDB) (https.//www.rcsb.org). Each structure represents a unique CAM chemistry. To compare these structures, helix 5 and the subsequent extended structure (residues 111-142) of the “pocket” subunit were aligned as shown in FIG. 1. In this alignment, the CAMs largely overlapped.

TABLE 1
Comparing CAM-HBc Complex Structures

| PDB | Cp | CAM |
|---|---|---|
| 7s76 | Y132A | AB506, tetrahydro-cyclopentapyroles |
| 5d7y | capsid | HAP, HAP18 |
| 5e0i | Y132A | NVR10-001 HAP |
| 5T2P | Y132A | SBA, sulfamoyl benzamide SBA_R01 |
| 5WRE | Y132A | HAP, HAP_R01 |
| 7K5M | Y132A | PPyr pyrazolopyridine |
| 5GMZ | Y132A | HAP |
| 6J10 | Y132A | cyclopyrox |
| 4G93 | capsid | PPA, Phenylproenamide AT130 |
| 6BVF | capsid | HAP, HAP-TAMRA |
| 6WFS | capsid | DBT, Dibenzothiazepine DBT1 |

The list of CAMs set out in Table 1 are exemplary and based upon CAMs with published structures of the CAM-Cp complex. The exemplary CAMs of Table 1 are provided to illustrate the overlap between the structure and their alignment within the “pocket” subunit. Those of skill in the art will appreciate that other CAMS described in the literature, as well as variations of the CAMs in Table 1, share this alignment and may also be utilized in the rCAMs described herein.

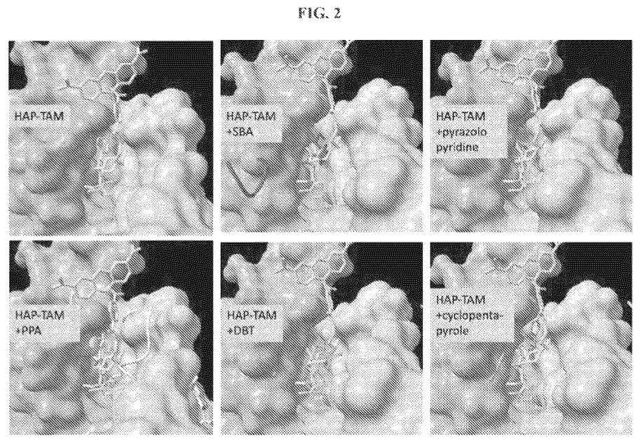

Because the CAM pocket geometry is defined by the contact between two dimers, CAMs follow the same binding pattern. This means that all CAMs can be extended into the lumen of a capsid allowing a molecule attached to the CAM entry to the capsid lumen. FIG. 2 provides an overlay for each of the CAMs listed in Table 1 and as aligned in FIG. 1. In order to emphasize the CAM structure in each of the images of FIG. 2, the protein subunits of the capsid are covered with a molecular surface, where the pocket subunit is positioned on the left (in red) and the capping subunit is positioned on the right (in green). Additionally, in each of the panels of FIG. 2, a CAM-linker-fluorophore construct (HAP-TAMAR) that structurally resembles a rCAM has been included to facilitate visualizing the positioning of the CAM. In each case, the bound CAM follows the path of HAP-TAMRA and thus identifies the best attachment point on the CAM for the rCAM linker.

As shown in FIG. 3, linker attachment points for several exemplary CAMs are shown (designated by a red circle). Exemplary CAMs include pyrazolopyridinyl sulfonamide, sulfonylpiperidine carboxamides, sulfamoyl benzamide, dibenzothiazapine, sulfamoyl benzamide 2, heteroaryldihydropyrimidine, pyrole benzamide, AT130 phenylpropenamide, tetrahydropyrazolopyrazine, diazepinone, ABI-2158, AB506 pyrole benzamide, and analogs thereof. The CAMs and designated attachment points shown in FIG. 3 are exemplary and are not an exhaustive list of the CAMS and attachment points that may be utilized in the rCAM structures disclosed herein. Those of skill in the art would appreciate that in many cases, the functional groups associated with an attachment point can be changed with nominal cost to efficacy, expanding the description of the structure-activity relationship (SAR). This is exemplified in patents describing the DBT family of CAMs (e.g. patents U.S. Pat. No. 10,183,936 B2 and WO2017/048950).

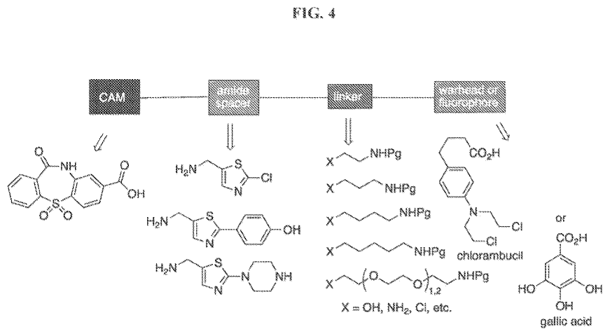

The CAMs are often combined with an amide-linked spacer in order to modify the binding affinity of the CAM in the pocket. For example, the CAM dibenzothiazepine (DBT) matched with amino-linked substituents has been described in U.S. Pat. Nos. 10,183,936, 10,947,224, 11,560,370, and 11,078,170, incorporated herein by reference. In one rCAM embodiment set out in FIG. 4, the rCAM is designed by combining the CAM molecule DBT with one amide spacer, one linker, and one warhead to create the final rCAM molecule.

The rCAMs disclosed herein are selected to localize the warhead to viral nucleic acid and provide an attachment point for a linker and warhead. This effect can be quantified. The interior of a capsid nominally has 120 or 240 CAM binding sites, depending on the CAM. See, e.g., Schlicksup, C. J., et al., ACS Chem Biol 15:1708-1717, 2020; Schliksup, C. J. and Zlotnick, A., Curr Opin Virol 45:43-50, 2020; and Schliksup, C. J., et al., Elife 7:pii:e31473. Consider that the capsid interior has a radius of 12.5 nm, the “effective” concentration of 120 CAMs per capsid is 24 mM. For a typical bulk concentration for a biologically active CAM of 100 nM, this means that the intra-capsid warhead concentration is 240,000-fold higher than the bulk concentration. This effective concentration allows a modestly active warhead to have a greater impact on the virus. Additionally, because the rCAM can have antiviral efficacy at a low concentration, the rCAM does not rely upon the capsid assembly and disassembly functions of the CAM to have antiviral efficacy. Furthermore, optimizing an rCAM for DNA and RNA destructive activity will generally require depressing CAM assembly and disassembly activity to increase the exposure of nucleic acid to the warhead.

### Linkers

The role of the linker in an rCAM is to extend the warhead beyond the confines of the CAM pocket. The linker must then be optimized for efficacy, depending on warhead mechanism, and other pharmacology (e.g., metabolic stability, bioavailability, safety). In one embodiment of an rCAM, where the warhead mechanism depends on collision with nucleic acid and the CAM component binds very tightly to the capsid, the linker must be long enough for the reactive moiety to reach the nucleic acid. In another embodiment, the CAM binds stochastically so that when the CAM is not bound to Cp, the reactive group can collide with nearby nucleic acid. In a third embodiment, where the reactive moiety generates reactive oxygen species (ROS) that diffuse away from the warhead, the linker does not have to reach the nucleic acid as the ROS (i.e., hydroxy radicals) generated by the reactive moiety can diffuse to, and react with, the nucleic acid.

A general formula for a linker can include reactive functional group1 (FG₁)-inert chain-reactive functional group2 (FG₂), where the reactive functional groups are chosen to facilitate attachments to the CAM and warhead functionalities. In one example, the linker may have the following general formula (I):

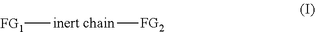

where the functional groups FG₁ and FG₂ are chosen to facilitate attachments to the CAM and warhead functionalities. In some embodiments, FG₁ and FG₂ may be independently selected from C₁₋₆alkyl, C₂₋₆alkenyl, C₁₋₆alkoxy, C₂₋₆alkynyl, or hydroxy. In some embodiments, the linker may include at least one amine group (—NH). In some embodiments, the amine groups do not repeat but are separated by an alkyl group. In preferred embodiments, the linker sequence begins and ends with an amine group. In additional preferred embodiments, the linker includes two amine groups (—NH), where each amine group is separated by an alkyl chain. The alkyl chain can include an ether functional group.

In some embodiments, the linker may include or have the formula amine-R-amine-, where R is an alkyl chain including at least one methylene (—CH₂) group. In some embodiments, the linker may be amine-R-amine, where R is an alkyl group of two to six methylene groups, two to five methylene groups, two to four methylene groups, or two to three methylene groups, wherein each range is inclusive.

In some embodiments, the linker may include or have the formula amine-R-amine, where R is an alkyl group of one to four ethylene glycols (i.e., PEG) sandwiched between two amines. Another example might be hydroxy-R-amine, or carboxyl-amine-carboxyl: amine-ethyl-R-ethyl-amine. Linkers may be straight or branched carbon chains. In some examples, the linker has a length of 0 to 20 angstroms (Å), 5 to 30 Å, 1 to 15 Å, 0 to 5 Å, 5 to 10 Å, 5 to 15 Å, or 15 to 20 Å, where each range is inclusive. Preferably, the linker is an organic chain of about 0-20 Å or 5-30 Å in length.

### Reactive Moiety (“Warhead”)

Disclosed rCAM compounds include reactive moieties, referred to herein also as “warheads,” which must have the ability to modify DNA. Among others, two general classes of reactive moieties useful in the disclosed rCAMS are chemotherapeutic molecules (such as mustards) and reactive oxygen species (ROS) generators. In some examples, rCAM compounds can exert antiviral activity by accelerating the assembly of flawed capsids. In other examples, rCAM compounds can exert antiviral activity by directly damaging DNA, e.g., by causing DNA breakage, by nicking DNA, and the like.

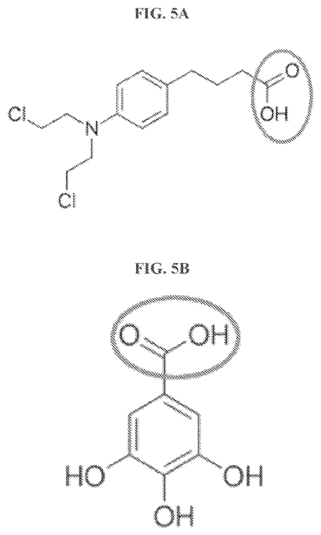

In some embodiments, the reactive moiety of disclosed rCAM compounds includes an alkylating agent. Many chemotherapy drugs are small molecules that can alkylate DNA, leading to adduct formation or cross-linking which can impair transcription or promote DNA breakage. Deans, A. J. and West, S. C., Nat Rev Cancer 11:467, 2011; Fu, D., et al., Nat Rev Cancer 12:104, 2012. It should be noted that many alkylating agents can chemically alter RNA as well. Chlorambucil is a classic bifunctional, nitrogen mustard that has been used to treat several different cancers. Mustards are often used as chemotherapeutics. They can alkylate DNA (e.g. on N7 of Guanine) or induce interstrand crosslinks and double stranded breaks in DNA. Chlorambucil is an exemplar of a mustard, approved for treatment of some lymphomas and other blood related cancers. The point of attachment of a linker to chlorambucil is circled in the structure illustrated in FIG. 5A.

Exemplary classes of alkylating agents include nitrogen mustards, nitrosoureas, alkyl sulfonates, triazines, and ethylenimines. Exemplary alkylating agents include bendamustine, chlorambucil, cyclophosphamide, ifosfamide, mechlorethamine, melphalan, carmustine, lomustine, streptozocin, busulfan, dacarbazine, temozolomide, altretamine, thiotepa, and analogs thereof.

In some embodiments, chemotherapeutic molecules for use as a reactive moiety in the rCAM molecules include, but are not limited to, altretamine, mitomycin C, cisplatin, phleomycin, and analogs thereof. Altretamine and Mitomycin C are known to cause G-G and G-A interstrand cross-linking. Cisplatin is another well-known cross-linking cancer therapeutic that targets DNA, but it can also chemically modify RNA (Hostetter, A. A., et al., J Am Chem Soc 131:9250, 2009). Phleomycin is a glycopeptide antibiotic that can bind to DNA and promote DNA cleavage (Stokowa-Soltys, K., et al., J Inorg Biochem 195:71, 2019). Those of skill in the art will appreciate that other small chemotherapeutic molecules and reactive moieties with the ability to modify DNA may be useful in the disclosed rCAM molecules.

In some embodiments, the reactive moiety of disclosed rCAM compounds includes a reactive oxygen species (ROS) generating agent, also referred to herein as an ROS generator. ROS generators can provoke oxidative stress and free radicals, thereby inducing DNA damage, inhibiting DNA repair, and indirectly inducing apoptosis. In one example, gallic acid (3,4,5-trihydroxybenzoic acid) is one of the smallest known ROS generators. See, e.g., Chen et al., Evid Based Complement Alternat Med. 2013; 2013: 613950. The point of attachment of a linker to gallic acid is circled in the structure illustrated in FIG. 5B. The ROS-generating activity of quercetin has also been described (Kim et al., Oncotarget. 2017 Sep. 12; 8(39): 64964-64973).

Exemplary classes of ROS-generating agents include phenolic compounds, quinones, flavonoids, and transition metals. Exemplary ROS-generating agents include gallic acid, quercetin, luteolin, and analogs thereof. Herein, “ROS-generating agents” may be referred to interchangeably as “ROS generators.”

Thus far, two general classes of reactive moieties have been described: ROS generators and alkylators. Alkylating agents such as mustards typically target an electron-rich site and require direct interaction with the target. ROS generators, such as gallic acid, are biologically active redox agents. They generate diffusible reactants. In contrast to alkylating agents, the ROS generator need not contact the target nucleic acid.

### Compositions

In some aspects, provided herein are pharmaceutical compositions comprising disclosed rCAM compounds. In some embodiments, the provided compositions can be used in connection with any of the methods described herein, e.g., methods of treatment.

In some embodiments, disclosed pharmaceutical compositions are provided as unit dose form compositions for administration of a given dose or fraction thereof.

The pharmaceutical compositions generally include one or more optional pharmaceutically acceptable carrier or excipient. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

Compositions for in vivo administration are generally sterile. Sterility may be readily accomplished, e.g., by filtration through a sterile filtration membrane.

Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine, preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Disclosed compositions may include buffering agents. Suitable buffering agents include, e.g., citric acid, sodium citrate, phosphoric acid, potassium phosphate, and various other acids and salts. In some examples, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

Disclosed compositions can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition.

Accordingly, there are a variety of suitable formulations. The pharmaceutical composition may also contain more than one active ingredients useful for the particular indication, disease, or condition being treated. In some embodiments, the pharmaceutical composition includes at least one additional therapeutic agent.

Disclosed compositions may be formulated for oral, intravenous, intraperitoneal, subcutaneous, pulmonary, transdermal, intramuscular, intranasal, buccal, sublingual, or suppository administration. In some embodiments, the disclose compositions are administered parenterally. The term “parenteral,” as used herein, includes intravenous, intramuscular, subcutaneous, rectal, vaginal, and intraperitoneal administration.

Disclosed compositions may be provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Various additives which enhance the stability and sterility of the disclosed compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate, and gelatin. Sustained-release preparations may also be prepared.

### EXAMPLES

### Example 1: Selection of Reactive Moieties for Exemplary rCAMs

To develop rCAMs that can target and modify genome-filled capsids, an activity test was performed with several known chemotherapeutics to determine the best candidates for coupling to CAMs. In vitro transcribed pregenomic HBV RNA (pgRNA) (~3.2 kb) was used as the viral genomic material for testing modification by drug molecules. The following molecules were tested: chlorambucil, mitomycin C, cisplatin, phleomycin, and atretamine.

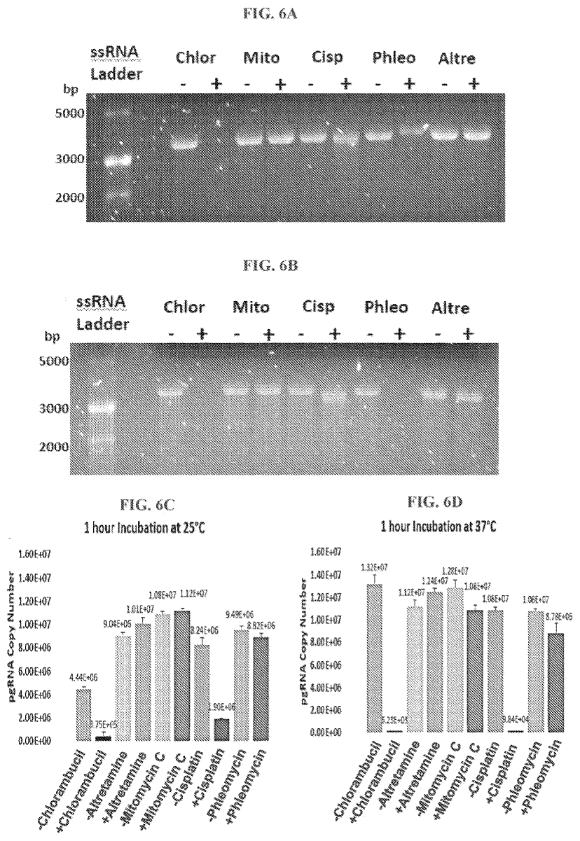

For the activity assay, pgRNA was incubated with the respective chemotherapeutic (900 μM) for 1 hour at 25° C. or 37° C. Then, the reaction samples were examined for evidence of alkylation via denaturing agarose gel electrophoresis (FIG. 6A and FIG. 6B) and RT-qPCR (FIG. 6C and FIG. 6D). For the electrophoresis experiments, it was predicted that RNA bands which migrated faster or decreased in thickness would signify evidence of cross-linking or cleavage respectively. Similar results were observed in both temperature conditions (FIG. 6A and FIG. 6B). Treatment with chlorambucil appeared to completely dissipate RNA at both temperatures, potentially indicating RNA cleavage. Addition of cisplatin led to faster migrating and smeared bands, indicating cross-linked RNA. For phleomycin, the pgRNA band appeared to be reduced in width at room temperature; at 37° C., the band completely disappeared. Reduction of the RNA band indicated cleavage, and higher temperature seemed to facilitate this reaction, probably by increasing the rate of collision between the antibiotic and pgRNA. For all other molecules, there appeared to be no differences between the control and treated RNA samples in either temperature conditions.

It is possible that alkylation by some of these molecules would be in the form of a simple monoadduct addition to the RNA, and gel electrophoresis would not be sensitive enough to detect this modification. For this reason, we turned to RT-qPCR because alkylation of the RNA template could impair transcription. For the RT-qPCR experiments, the Cp gene within the pgRNA was amplified to determine evidence of nucleic acid modification. At 25° C., incubation with chlorambucil and cisplatin led to approximately 1-log reduction in RNA amplification (FIG. 6C and FIG. 6D). Strikingly, incubation at 37° C. led to 4- and 3-log decreases in RNA levels by chlorambucil and cisplatin, showing that higher temperature facilitates more chemical modification. At either temperature, phleomycin does not suppress pgRNA amplification appreciably, even though the gel indicated RNA cleavage. It is possible that phleomycin cleaves the pgRNA in areas outside of the Cp gene, allowing for those fragments to still be transcribed. Consistent with gel electrophoresis, treatment with mitomycin C and altretamine does affect RNA transcription. From these results, chlorambucil and cisplatin were the most promising candidates for attaching to CAMs.

### Example 2: Synthesis of Exemplary rCAM HAP13-PEG-CBL

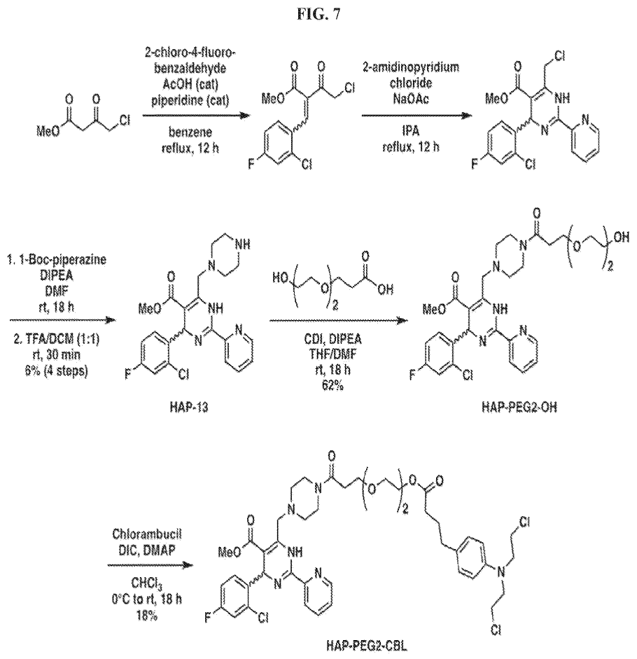

Two rCAM molecules having HAP13 as the CAM backbone and chlorambucil (CBL) as the reactive moiety were synthesized. The HAP13 and chlorambucil (CBL) were attached by PEG linkers of varying lengths: PEG2 (17.6 Å) and PEG4 (24.6 Å) (FIG. 7). The PEG linker lengths were chosen based on the reported distance between pgRNA and the internal capsid wall (~31 Å). (Wang, J. C., et al., PLoS Pathog 8:e1002919, 2012). It was predicted that the length of the PEG4 construct would be sufficient to probe the encapsidated pgRNA, and the PEG2 construct would be used as a control to test this concept.

### Methyl-4-(2-chloro-4-fluorophenyl)-6-(piperazin-1-ylmethyl)-2-(pyridin-2-yl)-1,4-dihydropyrimidine-5-carboxylate (HAP-13)

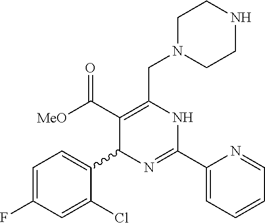 [SMILES: COC(=O)C1=C(CN2CCNCC2)NC(c2ccccn2)=NC1c1ccc(F)cc1Cl]

HAP 13 was synthesized via previously established methods to produce a yellow powdery solid. US 20150218182-A1; US 201602741112-A1. Rt=6.728 minutes [analytic RP HPLC (10% MeCN (aq) 0.1% TFA→90% MeCN(aq) 0.1% TFA linear gradient over 10 minutes, 1 mL/min, 254 nm); TOF-MS (ESI+) m/z [M+H]+ calculated for C₂₂H₂₃ClFN₅O₂ (M+H)+ 444.2; found 444.3.

### 1-hydroxy-3,6,9,12-tetraoxapentadecan-15-oic acid (Hydroxyl-PEG₂-COOH)

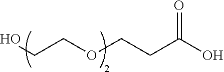

To a solution of hydroxyl-PEG2-tButyl ester (1.0 eq) in DCM (250 μL, 1 M) was added dropwise TFA (250 μL, 1 M). The reaction was stirred at room temperature for 1 hour, and then solvent was completely removed under reduced pressure. The crude colorless oil was taken to the next reaction without further purification.

### Methyl-4-(2-chloro-4-fluorophenyl)-6-((4-(3-(2-(2-hydroxyethoxy)ethoxy) propanoyl)piperazin-1-yl)methyl)-2-(pyridin-2-yl)-1,4-dihydropyrimidine-5-carboxylate (HAP-PEG2-OH)

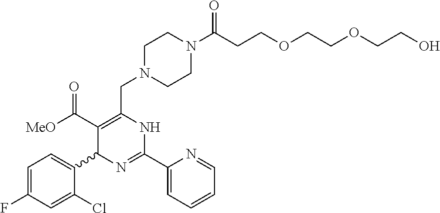 [SMILES: COC(=O)C1=C(CN2CCN(C(=O)CCOCCOCCO)CC2)NC(c2ccccn2)=NC1c1ccc(F)cc1Cl]

To a stirring solution of hydroxyl-PEG2-COOH (60 mg, 0.335 mmol) in THF (4.8 mL) was added CDI (54 mg, 0.335 mmol). Upon stirring for 2 hours, HAP-13 (114 mg, 0.258 mmol) was added, and the solution was aged overnight. Organic solvent was removed in vacuo, and the crude residue was dissolved in methanol, filtered, and concentrated to afford a yellow solid that was taken to the next step without further purification (96 mg, 62%). TOF-MS (ESI+) m/z [M+Na]+ calculated for C29H35ClFN5O6Na (M+Na)+ 626.2152; found 626.2154.

### Methyl 6-((4-(3-(2-(2-((4-(4-(bis(2-chloroethyl)amino)phenyl)butanoyl) oxy)ethoxy)ethoxy)propanoyl)piperazin-1-yl)methyl)-4-(2-chloro-4-fluorophenyl)-2-(pyridin-2-yl)-1,4-dihydropyrimidine-5-carboxylate (HAP-PEG2-CBL)

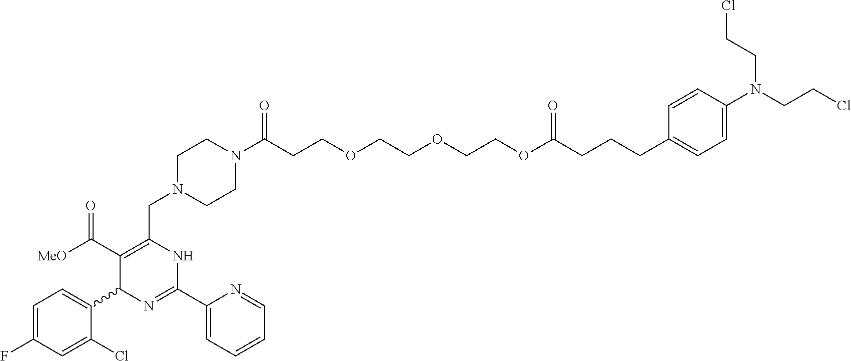 [SMILES: COC(=O)C1=C(CN2CCN(C(=O)CCOCCOCCOC(=O)CCCc3ccc(N(CCCl)CCCl)cc3)CC2)NC(c2ccccn2)=NC1c1ccc(F)cc1Cl]

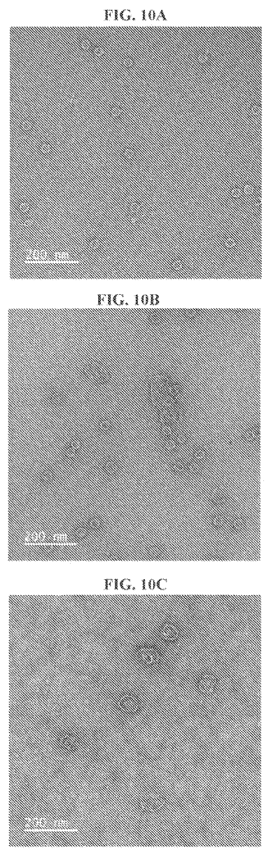

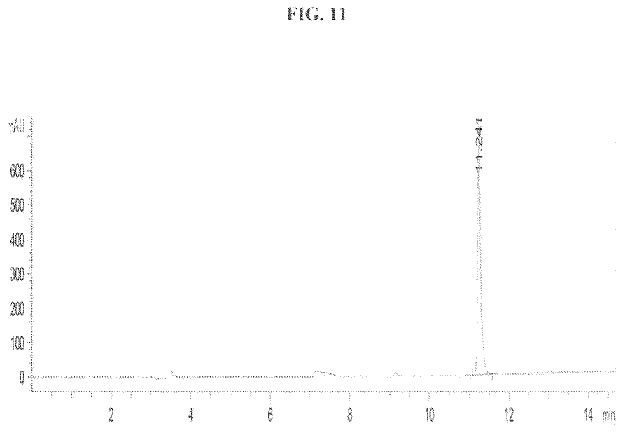

To a stirring solution of HAP-13 Hydroxyl-PEG4 (31 mg, 0.045 mmol) and chlolambucil (27 mg, 0.090 mmol) in DCM (225 L) at 0° C. was added DMAP (5 mg, 0.045 mmol) and DIC (14 μL, 0.090 mmol). The solution was warmed to room temperature and stirred overnight. The yellow mixture was concentrated in vacuo, and the crude residue was purified via preparative RP HPLC (10% MeCN (aq) 0.1% TFA→90% MeCN(aq) 0.1% TFA linear gradient over 10 minutes, 20 mL/min, 254 nm) to afford a yellow residue (17 mg, 18%). Analysis of the compound is shown in FIG. 10

The resulting rCAMs were designated HAP-PEG2-CBL and HAP-PEG4-CBL. The overall yield for HAP-PEG2-CBL and HAP-PEG4-CBL were 11% and 14% respectively.

### Example 3: Synthesis of Exemplary rCAM DBT-Linker-CBL

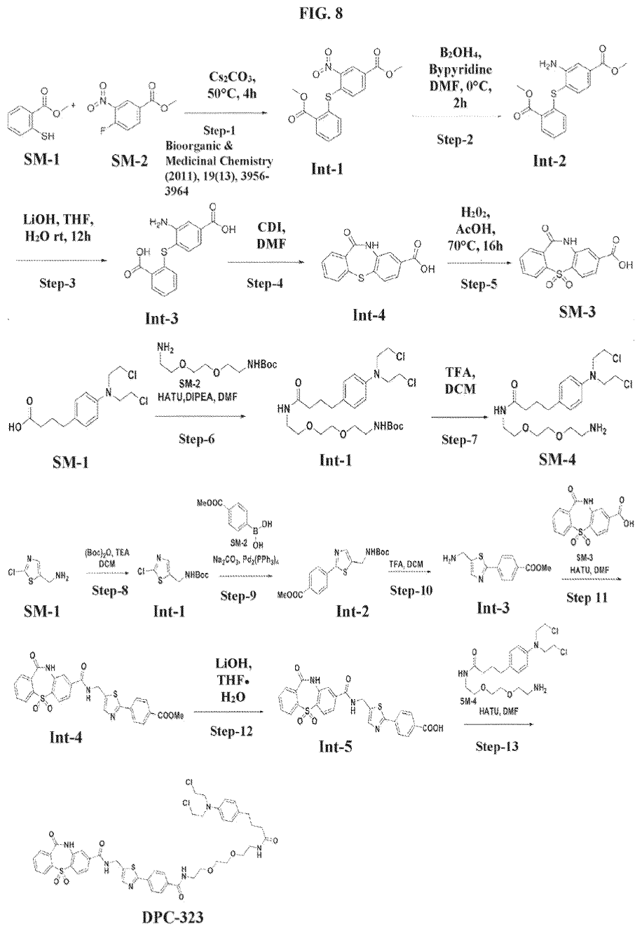

As shown in FIG. 8 another exemplary rCAM disclosed herein is one using Dibenzothiazepine (DBT) as the CAM module and chlorambucil (CBL) as the reactive moiety. Those of skill in the art will appreciate that the synthetic route for creating the exemplary rCAMs disclosed herein, such as the DBT-linker-CBL molecule, relies upon well-established amide bond coupling chemistries (such as Mitsunobu and/or nucleophilic displacement) for joining the CAM-amide space units as well as for the installation of the reactive moiety.

### Example 4: Verifying Functionalities of HAP-PEG-CBL Molecules

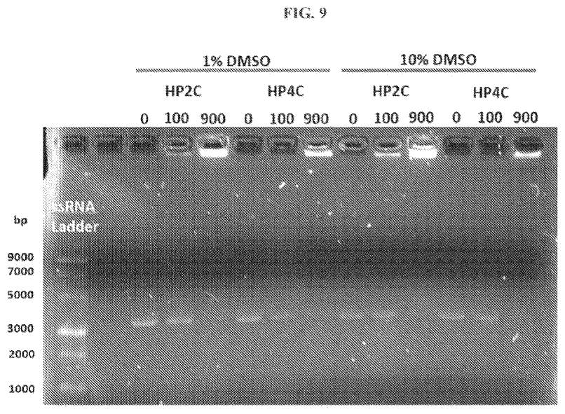

The two HAP-PEG-CBL molecules were tested to verify that they maintained their capsid-binding and alkylating functionalities. First, the gel electrophoresis experiment was repeated with the new constructs. Free pgRNA was titrated with either HAP-PEG2-CBL or HAP-PEG4-CBL in the following concentrations: 0, 100, and 900 μM and incubated at 37° C., for maximum effect (FIG. 9). DMSO concentrations were also tested to determine if solubility of the drugs impacted the molecule's alkylating effects. Reaction samples were prepared with a final DMSO concentration of 1% or 10% (v/v). For both HAP-PEG2-CBL and HAP-PEG4-CBL, 100 μM treatments led to slight reduction in RNA signal; drug treatment at 900 μM caused almost complete dissipation of RNA bands for both molecules. Drug incubation in 1% or 10% DMSO did not appear to affect the molecules' ability to modify the viral RNA.

### Example 5: Verifying Ability of HAP-PEG-CBL Molecules to Bind Cp Interface

Cp dimers were assembled with HAP-PEG2-CBL and HAP-PEG4-CBL to verify the HAP backbone's ability to bind the dimer-dimer interface. Cp149 dimers were mixed with each rCAM in a 1:5 ratio (dimer:rCAM). Assembly was induced by addition of NaCl (300 mM) followed by incubation overnight at 25° C. Assembly products were visualized via transmission electron microscopy (TEM) (FIG. 10). Assembly in NaCl alone led to normal spherical capsids (FIG. 10A). When assembled with HAP-PEG2-CBL and NaCl, Cp form both spherical capsids, faceted/broken capsids, and flat, aberrant structures (FIG. 10B); these misassembled particles resemble those previously observed with other HAPs. Boume, C., et al., J Virol 82:10262, 2008. Assembly with HAP-PEG4-CBL and NaC led to formation of floret-shaped, aberrant particles (FIG. 10C). From the gel electrophoresis and TEM data, both HAP-PEG2-CBL and HAP-PEG4-CBL exhibit capabilities for interrogating nucleic acid-filled capsids.

### Example 6: Further Characterization of rCAM Antiviral Effects

In biological assays, the antiviral effect of the CAM and the antiviral effect of the reactive moiety may be difficult to separate. It is discouraged to have a highly active CAM but not the desired DNA-destroying capabilities of the rCAMs described herein. Therefore, in addition to determining the rCAMs ability to modulate assembly and bind Cp as described in the Example above, targeted nucleic acid damage is also evaluated. Capsids are treated with the rCAM, RNA is then extracted and evaluated for defects by RT-qPCR (of the core gene), which is sensitive to breaks and lesions.

Additionally, the antiviral effects of CAM-alkylators in HBV infected cells are tested in infected HepG2-NTCP cells. Treatment shortly after infection tests for both CAM-induced disruption of capsid and alkylator-induced damage to the genome. Treatment in cells with established infection tests for the ability of the CAM-alkylator to be directed to established cccDNA.

To test rCAMs for the ability to prevent DNA deposition in the nucleus, rCAM treatment begins <3 h post-infection (p.i). At 5 d p.i., the supernatant is tested for HBeAg expression by ELISA. HBeAg is a Cp variant, expressed from the same gene as Cp but from an earlier start codon that is secreted from infected cells.

To test for destruction of viral nucleic acid in new virion, rCAM treatment is started 24 h p.i. At 5 d p.i., the media is tested for viral DNA by qPCR. Here the cccDNA is established, so the decrease in virus production is due to damage to encapsidated nucleic acid or to cccDNA.

### Example 7: Synthesis of Exemplary rCAM DP41-0012 (N-(3-cyano-4-fluorophenyl)-1-methyl-4-(N-(2-(3,4,5-trihydroxybenzamido)ethyl)-sulfamoyl)-1H-pyrrole-2-carboxamide)

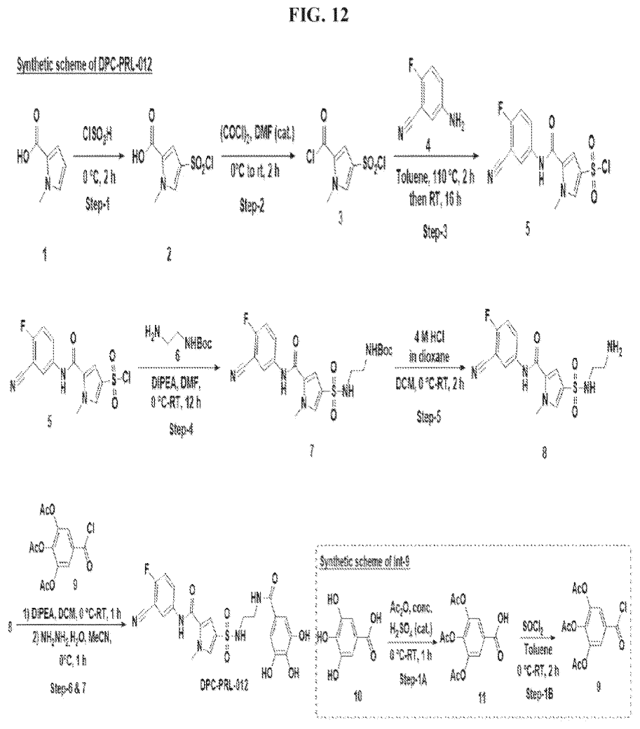

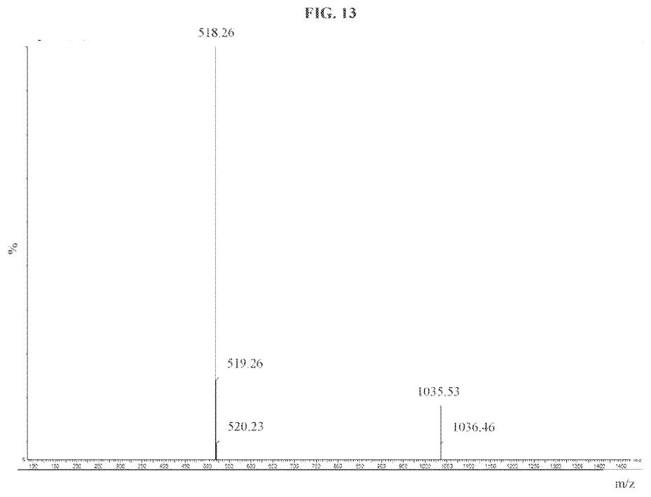

Exemplary rCAM DP41-0012 was synthesized according to the following methods, as shown in FIG. 12. DP41-0012 has a pyrrole-2-carboxamide CAM structure linked to a gallic acid nucleic acid disrupting reactive moiety. The structure of DP41-0012 is shown below.

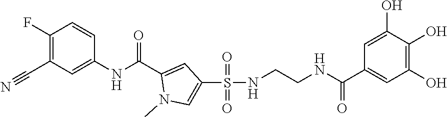 [SMILES: Cn1cc(S(=O)(=O)NCCNC(=O)c2cc(O)c(O)c(O)c2)cc1C(=O)Nc1ccc(F)c(C#N)c1]

Step-1: Chlorosulfonic acid (16 mL, 240.31 mmol) was cooled to 0° C. and then 1-methylpyrrole-2-carboxylic acid 1 (3 g, 23.98 mmol) was added in portions. The resulting reaction mixture was stirred at 0° C. for 2 h. The progress of the reaction was monitored by TLC. After completion of the reaction, the reaction mixture was quenched with ice-cold water and stirred for 1 h. The solid precipitated was filtered and dried to afford 4-chlorosulfonyl-1-methyl-pyrrole-2-carboxylic acid 2 (3.5 g, 65.2% yield) as a brown solid.

Compound 2 ¹H NMR characterization: 1H NMR (400 MHz, DMSO-d₆) δ=7.11 (s, 1H), 6.72 (s, 1H), 3.78 (s, 3H)

Steps 2 & 3: To a stirred solution of 4-(chlorosulfonyl)-1-methyl-1H-pyrrole-2-carboxylic acid 2 (3 g, 13.42 mmol) in dichloromethane (20 mL) at 0° C., oxalyl chloride (1.2 mL, 13.99 mmol) was added and the resulting reaction mixture was stirred at 0° C. for 2 h. The progress of the reaction was monitored by TLC (by quenching with methanol thus forming methyl ester). After completion of the reaction, the reaction mixture was concentrated under reduced pressure, the residue obtained was co-distilled with dry toluene and dried to afford 4-chlorosulfonyl-1-methyl-pyrrole-2-carbonyl chloride 3 (3.1 g, 13 mmol, 95% yield) as a brown solid (Note: The product obtained was taken as such to the next step without any purification).

To a stirred solution of 4-chlorosulfonyl-1-methyl-pyrrole-2-carbonyl chloride 3 (3.1 g, 13 mmol) in dry toluene (30 mL, 281 mmol) at 0° C., 5-amino-2-fluoro-benzonitrile 4 (2.6 g, 19.1 mmol) was added under nitrogen atmosphere. The resulting reaction mixture was stirred at 110° C. for 2 h and then at RT for 16 h. The progress of the reaction was monitored by TLC. After completion of the reaction, the reaction mixture was filtered, the filtrate obtained was concentrated under reduced pressure to afford 5-[(3-cyano-4-fluoro-phenyl)carbamoyl]-1-methyl-pyrrole-3-sulfonyl chloride 5 (3.2 g, 6.0 mmol, 47% yield) as a brown solid.

Compound 5 LC-MS characterization: LC-MS: 63.83% at 1.50 min; m/z=340.07 [M−H]⁻ [Column: BAKERBOND Q2100 C18 (2.1×50 mm, 1.8 m); Mobile phase A: 0.05% formic acid in water, Mobile phase B: 0.05% formic acid in MeCN; Gradient B %: 0.0/2_0.3/2_2.0/98_2.8/98_3.0/2_3.7/2; Flow rate: 0.6 mL/min (gradient)]

Step 4: To stirred solution of 5-((3-cyano-4-fluorophenyl)carbamoyl)-1-methyl-1H-pyrrole-3-sulfonyl chloride 5 (800 mg, 2.34 mmol) in DMF (4 mL) at 0° C., were added N,N-diisopropylethylamine (1.64 mL, 9.39 mmol) and tert-butyl (2-aminoethyl)carbamate 6 (451 mg, 2.82 mmol) and the resulting reaction mixture was stirred at room temperature for 12 h. The progress of the reaction was monitored by TLC. After completion of the reaction, the reaction mixture was quenched with ice-cold water and extracted with 10% methanol in dichloromethane. The organic phase was dried over anhydrous sodium sulphate, filtered, and concentrated under reduced pressure. The crude product obtained was purified by normal phase column chromatography (silica gel, 230-400 mesh) using eluent 65% ethyl acetate in heptane to afford tert-butyl (2-((5-((3-cyano-4-fluorophenyl)carbamoyl)-1-methyl-1H-pyrrole)-3-sulfonamido)ethyl)carbamate 7 (500 mg, 0.99 mmol, 42.67% yield) as a white solid.

### Compound 7 Physical Characterization:

LC-MS: 93.65% at 2.1 min; m/z=466.31 [M+H]⁺ [Column: BAKERBOND Q2100 C18 (2.1×50 mm, 1.8 μm); Mobile phase A: 0.05% formic acid in water, Mobile phase B: 0.05% formic in MeCN; Gradient B %: 0.0/2, 0.3/2, 2.0/98, 2.8/98, 3.0/2, 3.7/2; Flow rate: 0.6 mL/min (gradient)]

¹H NMR (400 MHz, DMSO-d₆) δ=10.37 (brs, 1H), 8.21 (d, J=3.5 Hz, 1H), 8.00 (d, J=3.9 Hz, 1H), 7.59 (s, 1H), 7.53 (t, J=8.8 Hz, 1H), 7.33 (s, 1H), 7.30-7.27 (m, 1H), 6.79 (brs, 1H), 3.92 (s, 3H), 3.04-2.97 (m, 2H), 2.84-2.77 (m, 2H), 1.34 (s, 9H)

Step 5: To a stirred solution of tert-butyl (2-((5-((3-cyano-4-fluorophenyl)carbamoyl)-1-methyl-1H-pyrrole)-3-sulfonamido)ethyl)carbamate 7 (500 mg, 1.07 mmol) in dichloromethane (5 mL) at 0° C., was added HCl (2 mL, 8 mmol, 4 M in 1,4-dioxane) and the resulting reaction mixture was stirred at RT for 2 h. The progress of the reaction was monitored by TLC. After completion of the reaction, the reaction mixture was concentrated under reduced pressure to obtain crude. The crude was triturated with diethyl ether followed by n-pentane and dried to afford 4-(N-(2-aminoethyl)sulfamoyl)-N-(3-cyano-4-fluorophenyl)-1-methyl-1H-pyrrole-2-carboxamide hydrochloride 8 (380 mg, 1.02 mmol, 88% yield) as an off-white solid.

### Compound 8 Physical Characterization:

LC-MS: 98.42% at 0.99 min; m/z=366.17 [M+H]⁺ [CORTECS, 3.0×30 mm, 1.6 um]; Mobile phase A: 0.05% formic acid in water, Mobile phase B: 0.05% formic acid in acetonitrile; (Time/B %): 0/3, 0.1/3, 1.4/97, 2/97, 2.05/3, 2.5/3, Flow rate: 0.85 mL/min (Gradient)]

¹H-NMR (400 MHz, DMSO-d₆) δ=10.51 (brs, 1H), 8.25 (d, J=3.1 Hz, 1H), 8.08-8.03 (m, 1H), 7.92 (brs, 3H), 7.65 (s, 1H), 7.59-7.52 (m, 2H), 7.42 (s, 1H), 3.93 (s, 3H), 3.07-2.99 (m, 2H), 2.89 (brs, 2H)

Steps 6 and 7: To stirred suspension of 4-(N-(2-aminoethyl)sulfamoyl)-N-(3-cyano-4-fluorophenyl)-1-methyl-1H-pyrrole-2-carboxamide hydrochloride 8 (100 mg, 0.25 mmol) in dichloromethane (3 mL) at 0° C., were added N,N-diisopropylethylamine (0.13 mL, 0.746 mmol) and (2,3-diacetoxy-5-chlorocarbonyl-phenyl) acetate 9 (94 mg, 0.299 mmol) and the resulting reaction mixture was stirred at room temperature for 1 h. The progress of the reaction was monitored by TLC. After consumption of the starting material, the reaction mixture was concentrated under reduced pressure to afford [2,3-diacetoxy-5-[2-[[5-[(3-cyano-4-fluoro-phenyl)carbamoyl]-1-methyl-pyrrol-3-yl]sulfonylamino]ethylcarbamoyl]phenyl]acetate (180 mg, crude). This crude (180 mg) was dissolved in acetonitrile (2 mL), cooled to 0° C., and hydrazine hydrate solution (0.075 mL, 1.10 mmol, 75% solution in water) was added into it. The resulting reaction mixture was stirred at 0° C. for 1 h. After completion of the reaction, the reaction mixture was concentrated under reduced pressure. The obtained crude product was purified by preparative HPLC [Column: X-SELECT CSH C18 (250×30 mm, 5 u); Mobile phase A: 0.1% formic acid in water, Mobile phase B: 100% acetonitrile; Flow rate 22 mL/min; Gradient [Time/% B: 0/5, 5/5, 12/25, 37/0, 40/98]) and the product fractions were lyophilized to afford N-(3-cyano-4-fluorophenyl)-1-methyl-4-(N-(2-(3,4,5-trihydroxybenzamido)ethyl)-sulfamoyl)-1H-pyrrole-2-carboxamide DP41-0012 (39.44 mg, 30.63% yield) as an off-white solid.

### Physical Characterization of DP41-0012:

LC-MS: 99.45% at 1.75 min; m/z=518.26 [M+H]⁺ [BAKERBOND Q2100 C18 (2.1×50 mm, 21.8 um), Mobile phase A: 0.05% formic acid in H₂O: acetonitrile (95:5), Mobile phase B: 0.05% formic acid in acetonitrile, Time/% B: 0.0/2, 0.3/2, 2.0/98, 2.8/98, 3.0/2, 3.7/2, Flow rate: 0.6 mL/min (Gradient)]

¹H-NMR (400 MHz, DMSO-d₆) δ=10.34 (brs, 1H), 8.97 (brs, 2H), 8.68 (brs, 1H), 8.21 (dd, J=6.0, 2.8 Hz, 1H), 8.05 (t, J=5.6 Hz, 1H), 8.02-7.96 (m, 1H), 7.58 (t, J=1.6 Hz, 1H), 7.52 (t, J=9.2 Hz, 1H), 7.40-7.32 (m, 2H), 6.79 (s, 2H), 3.89 (s, 3H), 3.37-3.24 (m, 2H), 2.96-2.86 (m, 2H)

As shown in FIG. 12, the synthesis of Int 9 was performed according to the following methods. Step-1 Å: To a stirred solution of 3,4,5-trihydroxybenzoic acid 10 (3.7 g, 22 mmol) in acetic anhydride (12 mL) at 0° C., conc. sulfuric acid (0.1 mL) was added, and the resulting reaction mixture was stirred at RT for 1 h. The reaction mixture was cooled to 0° C., H₂O (100 mL) was added, and the mixture was stirred at RT for 2.5 h. The solid precipitated was filtered and dried to afford 3,4,5-triacetoxybenzoic acid 11 (5.2 g, 80.7% yield) as an off-white solid.

Compound 11 ¹H-NMR Characterization: ¹H-NMR (400 MHz, DMSO-d₆) δ=13.25 (brs, 1H), 7.74 (s, 2H), 2.33 (s, 3H), 2.29 (s, 6H)

Step-1B: To a stirred solution of 3,4,5-triacetoxybenzoic acid 11 (1 g, 3.38 mmol) in toluene (8 mL) at 0° C., was added thionyl chloride (0.74 mL, 10.1 mmol) slowly and the reaction mixture was stirred at RT for 2 h. The progress of the reaction was monitored by TLC. After the completion of the starting material, the reaction mixture was concentrated under reduced pressure. The residue obtained was co-distilled with toluene followed by dichloromethane and dried to afford (2,3-diacetoxy-5-chlorocarbonyl-phenyl) acetate 9 (980 mg, 92.24% yield) as a white solid. It was used for the next step without further purification.

### Example 8: Capsid Assembly Assay

Stimulation of HBV virus assembly is a known property of CAM molecules. As structural changes can have unpredictable effects on function, a capsid assembly assay was conducted with exemplary rCAM DP41-0012 to evaluate whether the construct retained CAM activity. Relative peak HBV capsid volume following exposure to the exemplary rCAM was determined in comparison to dimer, DMSO, and capsid controls, along with CAM compounds bersacapavir and NVR-3-778.

To test capsid assembly, samples or controls were incubated for 20 minutes with purified capsid protein assembly domain, Cp149, so that final conditions were 10 μM Cp149 dimer 20 μM compound or control 2% DMSO 50 mM TrisHCl pH 7.5. To this mixture was added an equal volume of 600 mM NaCl 50 mM TrisHCl pH 7.5. Reactions were equilibrated for 24 h at room temperature and the resolved by size exclusion chromatography using a 10/300 Superose6 column running at 0.5 ml/minute. The running buffer for this column was 50 mM TrisHCl 300 mM NaCl. Elution profiles were recorded by absorbance at 280 nm. Integrated capsid peaks eluting between 16 and 20 minutes and dimer peaks eluting between 34 and 39 minutes were reported.

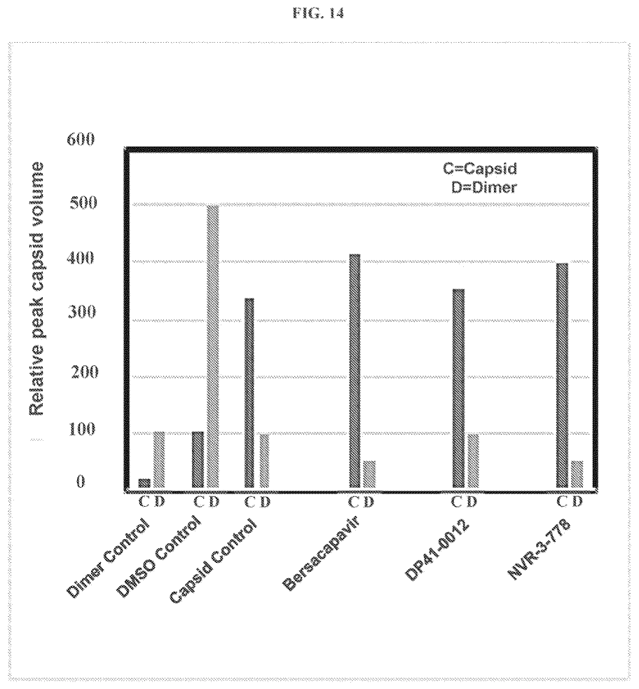

As shown in FIG. 14, exposure to CAM compounds and the exemplary rCAM compound DP41-0012 resulted in relative peak capsid volume that exceeded that of the capsid control. The results indicate that the exemplary rCAM compound retained HBV capsid stimulating activity associated with the included CAM molecule.

### Example 9: DNA Nicking Activity of rCAM Assay

The capacity of rCAM compounds to cause DNA damage was evaluated using an in vitro DNA nicking assay. Specifically, the double stranded DNA (dsDNA) nicking activity of exemplary rCAM DP41-0012 was compared to CAM compounds bersacapavir and NVR-3-778, and DMSO controls.

Supercoiled plasmid, the substrate for this assay, was purified from E. coli, using a Qiagen miniprep kit. A “master mix” of 3 ng/μl Miniprep DNA, 10 μM CuCl2, 20 mM TrisHCl pH7.5 was prepared. Samples included 99 μl of master mix plus 1 μl of compound or control. Reactions were incubated for 3 hours at room temperature (21° C.) in the dark. Aliquots of the reactions (20 μl) were resolved on a 0.8% TAE agarose gel with 1/20,000 SYBR Safe stain electrophoresed at 80V for 90 minutes. The gel was visualized on a ChemidocMP in fluorescein blot mode.

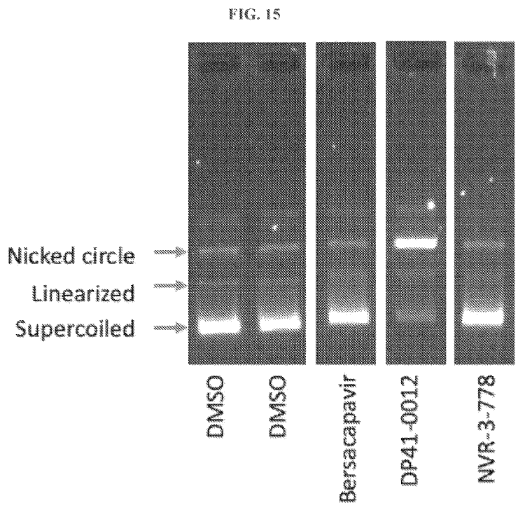

As shown in FIG. 15, the supercoiled plasmid substrate was detectable following exposure to DMSO control samples and CAM compounds bersacapavir and NVR-3-778. In contrast, exposure to exemplary rCAM DP41-0012 led to the observance of a DNA band corresponding to the size of a nicked circle. Together, these data show the DNA nicking activity of the exemplary rCAM, indicating an additional mechanism for damaging HBV DNA.

### EQUIVALENTS AND SCOPE

Those skilled in the art will recognize or be able to ascertain using no more than routine experimentation many equivalents to the specific embodiments described herein. The scope of the present invention is not intended to be limited to the above, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, it is to be understood that the invention encompasses all variations, combinations, and permutations in which one or more limitations, elements, clauses and descriptive terms, from one or more of the listed claims is introduced into another claim. For example, any claim that is dependent on another claim can be modified to include one or more limitations found in any other claim that is dependent on the same base claim.

Where elements are presented as lists, e.g., in Markush group format, it is to be understood that each subgroup of the elements is also disclosed, and any element(s) can be removed from the group. It should be understood that, in general, where the invention, or aspects of the invention is/are referred to as comprising particular elements, features, etc., certain embodiments of the invention or aspects of the invention consist, or consist essentially of, such elements, features, etc. For purposes of simplicity, those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranged can assume any specific value or sub-range within the stated ranges in different embodiments of the invention, to the tenth of the unit of the lower limit of the range, unless the context clearly dictates otherwise.

The term “about” or “approximately” means within an acceptable error range for the particular value as determined by one of the ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5% or up to 1% of a given value. Alternatively, the term can mean within an order of magnitude, for example within 5-fold, or within 2-fold, of a value. Where particular values are described in the application and claims, unless otherwise stated the term “about” meaning within an acceptable error range for the particular value should be assumed.

In addition, it is to be understood that any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the method of the invention can be excluded from any one or more claims, for any reason, whether or not related to the existence of prior art.

Each of the foregoing patents, patent applications, and references is hereby incorporated by reference, particularly for the teaching referenced herein.

Sequence Table

| SEQ | | |
|---|---|---|
| ID | | |
| NO. | Sequence | Description |
| 1 | MDIDPYKEFGATVELLSFLPSDFFPSV | Cp183 |
| | RDLLDTAAALYRDALESPEHCSPHHTA | |
| | LRQAILCWGDLMTLATWVGTNLEDPAS | |
| | RDLVVSYVNTNVGLKFRQLLWFHISCL | |
| | TFGRETVLEYLVSFGVWIRTPPAYRPP | |
| | NAPILSTLPETTVVRRRGRSPRRRTPS | |
| | PRRRRSQSPRRRRSQSRESQC | |
| 2 | MDIDPYKEFGATVELLSFLPSDFFPSV | Cp143 |
| | RDLLDTAAALYRDALESPEHCSPHHTA | |
| | LRQAILCWGDLMTLATWVGTNLEDPAS | |
| | RDLVVSYVNTNVGLKFRQLLWFHISCL | |
| | TFGRETVLEYLVSFGVWIRTPPAYRPP | |
| | NAPILSTLPETTVV | |

I claim:

1. A compound for directed delivery of HBV antiviral agent to HBV nucleic acid bound to capsid protein, comprising:
a) an HBV capsid assembly modulator (CAM);
b) a linker; and
c) a nucleic acid disrupting reactive moiety;
wherein the linker connects the CAM and the reactive moiety.

2. The compound of claim 1, wherein the CAM interacts with residues W102, S121, and T128 of the capsid protein.

3. The compound of claim 1, wherein the linker has a length of about 0 to 20 Å, inclusive.

4. The compound of claim 1, wherein the linker is of the following formula (I): 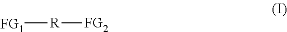
wherein FG₁ and FG₂ may independently comprise COOH, OH, and NH₂ and R comprises an alkyl group of 2 to 5 methylenes, inclusive.

5. The compound of claim 1, wherein the linker is of the following formula (I): 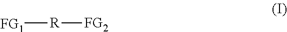
wherein FG₁ and FG₂ may independently comprise COOH, OH, and NH₂ and R comprises 1 to 6 polyethylene glycol units, inclusive.

6. The compound of claim 1, further comprising an amide spacer positioned between the CAM and the linker.

7. The compound of claim 1, wherein the CAM is connected directly to the nucleic acid disrupting reactive moiety.

8. The compound of claim 1, wherein the nucleic acid disrupting reactive moiety is selected from an alkylating agent or a reactive oxygen species generator.

9. The compound of claim 8, wherein the alkylating agent is a chemotherapeutic molecule selected from the group consisting of chlorambucil, altretamine, mitomycin C, cisplatin, phleomycin, and analogs thereof.

10. The compound of claim 8, wherein the reactive oxygen species generator comprises a polyphenol, a dihydroxy phenol, a trihydroxy phenol, proteocatachuic acid, gallic acid, 2,3,4-trihydroxybenzoic acid, a dihydroxybenzoic acid, or a combination thereof.

11. The compound of claim 10, wherein the dihydroxybenzoic acid comprises 2,3-dihydroxybenzoic acid, 2,4-dihydroxybenzoic acid, 2,5-dihydroxybenzoic acid, 2,6-dihydroxybenzoic acid, 3,4-dihydroxybenzoic acid, 3,5-dihydroxybenzoic acid, or a combination thereof.

12. The compound of claim 1, wherein the CAM is selected from the group consisting of tetrahydropyrazolopyrazine, sulfamoyl benzamide (SBA), pyrazolopyridine, phenylpropenamide (PPA), heteroaryldihydropyrimidine (HAP), pyrole benzamide, sulfonylpiperdine carboxamide, dibenzothiazepine (DBT), pyrrole carboxamides, and analogs thereof.

13. The compound of claim 1, wherein the compound comprises

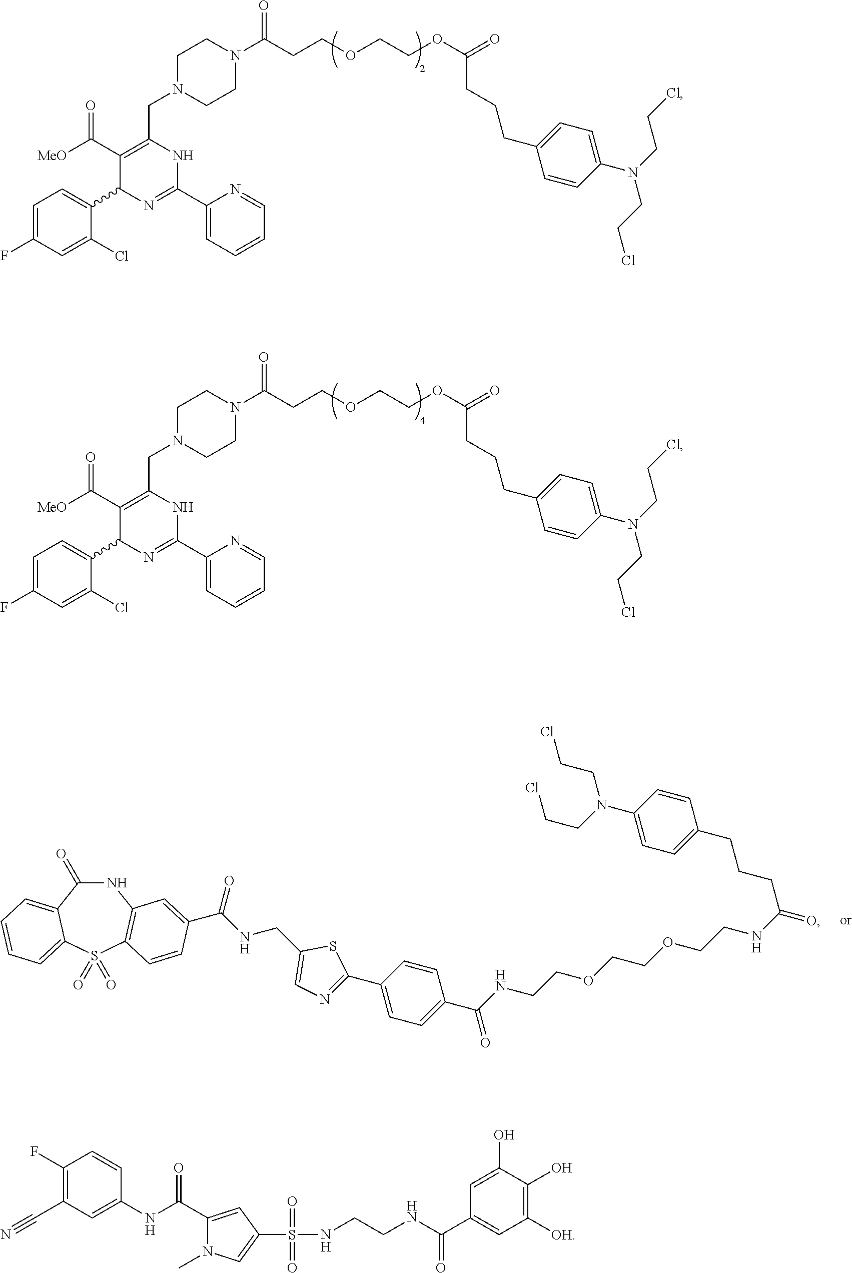

14. A pharmaceutical composition comprising the compound of claim 1 and a pharmaceutically acceptable excipient.

15. A method of making a compound for directed delivery of HBV antiviral agent to HBV nucleic acid bound to capsid protein, comprising:
a) coupling an HBV capsid assembly modulator (CAM) to a first end of a linker, and
b) attaching the nucleic acid disrupting reactive moiety to a second end of the linker, wherein the linker separates the CAM from the nucleic acid disrupting reactive moiety.

16. The method of claim 15, wherein the coupling step comprises an amide coupling reaction.

17. The method of claim 15, wherein the attaching step comprises an esterification reaction.

18. The method of claim 15, wherein the linker is absent, and the CAM is directly attached to the nucleic acid disrupting reactive moiety.
